(19) United States
(12) Patent Application Publication
(10) Pub. No.: US 2013/0209376 A1
(43) Pub. Date: Aug. 15, 2013

(54) ORAL CARE COMPOSITIONS

(71) Applicants: William E. Prosise, Ramsey, NJ (US); Robert J. Corring, Rockaway Township, NJ (US)

(72) Inventors: William E. Prosise, Ramsey, NJ (US); Robert J. Corring, Rockaway Township, NJ (US)

(73) Assignee: IPS Investments Inc., Wilmington, DE (US)

(21) Appl. No.: 13/640,962

(22) PCT Filed: Mar. 31, 2011
(86) PCT No.: PCT/US11/30642

§ 371 Date: Dec. 4, 2012

Related U.S. Application Data
(60) Provisional application No. 61/324,116, filed on Apr. 14, 2010.

(51) Int. Cl. A61K 8/81; A61Q 11/00

(52) U.S. Cl. CPC A61K 8/8176; A61Q 11/00
USPC 424/52; 424/49; 424/57

(57) ABSTRACT

Oral care compositions containing an oral care ingredient and a thickening agent, wherein the thickening agent comprises a strongly swellable, lightly to moderately crosslinked polyvinyl pyrrolidone. The polyvinyl pyrrolidone thickening agent is characterized by an aqueous gel volume of about 15 to 150 ml/g and a Brookfield viscosity of at least 10,000 cps for a 5% aqueous solution at 25° C.

### FIELD

The present application relates to oral care compositions, and, more particularly, to oral care compositions containing a strongly swellable, lightly to moderately crosslinked polyvinyl pyrrolidone.

### BACKGROUND

Oral care compositions are well known in the art. See, e.g., U.S. Pat. Nos. 5,288,480 and 6,723,304, and U.S. Pat. App. Pub. No. 2009/0269287, the disclosures of which are hereby incorporated by reference. Oral care compositions typically include an oral care ingredient and a thickening agent. The thickening agent is used to develop the desired viscosity for the oral composition so that the composition is suitable for its intended use.

### SUMMARY

The present application relates to compositions containing an oral care ingredient and a thickening agent, wherein the thickening agent comprises a strongly swellable, lightly to moderately crosslinked polyvinyl pyrrolidone. The polyvinyl pyrrolidone thickening agent is characterized by an aqueous gel volume of about 15 to 150 ml/g and a Brookfield viscosity of at least 10,000 cps for a 5% aqueous solution at 25° C.

In accordance with one embodiment of the present invention, the composition is an oral care composition containing at least one oral care ingredient. The oral care composition as described herein may take the form of a paste, gel or liquid. In accordance with particular aspects of the present invention, the oral care ingredient may be a polishing agent, a fluoride ion source, a humectant, a surface active agent, an anticaries agent, an anticalculus agent or combinations thereof.

In accordance with another aspect, a method of producing a stable oral care composition is disclosed. The method comprises adding a thickening agent comprising a strongly swellable, lightly to moderately crosslinked polyvinyl pyrrolidone to a base composition containing an oral care ingredient. In accordance with another method, the thickening agent is dispersed in a solvent such as water and then the oral care ingredients are added to the admixture of thickening agent and solvent.

### DETAILED DESCRIPTION

The present application relates to compositions containing an oral care ingredient and a thickening agent, wherein the thickening agent comprises a strongly swellable, lightly to moderately crosslinked polyvinyl pyrrolidone. More particularly, the present application discloses oral care compositions in the form of a gel, paste or liquid containing the lightly to moderately crosslinked polyvinyl pyrrolidone thickening agent. The oral care composition may be in various forms including toothpaste, dentifrice, tooth gel, subgingival gel, mouth rinse, mousse, foam, denture product, mouthspray, lozenge, chewable tablet or chewing gum. The oral care composition may also be incorporated onto strips or films for direct application or attachment to oral surfaces.

Various active oral care ingredients that can be utilized according to the present invention include conventional oral care ingredients such as polishing agents, fluoride ion sources, humectants, surface active agents, anticaries agents, and anticalculus agents. Mixtures of any of the foregoing active oral care ingredients are also suitable for use in the present invention.

The compositions described herein also include a thickening agent comprising a strongly swellable, lightly to moderately crosslinked polyvinyl pyrrolidone such as those described in commonly assigned U.S. Pat. Nos. 5,139,770 and 5,312,619, which are incorporated herein by reference. These thickening agents typically contain from about 0.25% to about 1% by weight of a crosslinking agent selected from the group consisting of divinyl ethers and diallyl ethers of terminal diols containing from about 2 to about 12 carbon atoms, divinyl ethers and diallyl ethers of polyethylene glycols containing from about 2 to about 600 units, dienes having from about 6 to about 20 carbon atoms, divinyl benzene, vinyl and allyl ethers of pentaerythritol, and the like.

The term “strongly swellable, lightly to moderately crosslinked PVP”, unless otherwise noted, specifically refers to polymer essentially consisting of lightly- to moderately-crosslinked poly(N-vinyl-2-pyrrolidone) having at least one of the following characteristics: (1) an aqueous swelling parameter defined by its gel volume from about 15 mL/g to about 300 mL/g, more particularly from about 15 mL/g to about 250 mL/g, and in other cases from about 15 mL/g to about 150 mL/g, or (2) a Brookfield viscosity of 5% crosslinked PVP in a liquid carrier comprising water at 25° C. of at least 2,000 cP, more particularly of at least about 5,000 cP, and in certain cases of at least about 10,000 cP. Disclosure for these parameter ranges is provided in U.S. Pat. No. 5,073,614 (incorporated herein by reference) and in Shih, J. S., et al. (1995). Synthesis methods for the crosslinked PVP are disclosed in a number of references, including U.S. Pat. Nos. 5,073,614; 5,654,385; and 6,177,068, the contents of which are hereby incorporated by reference. It is appreciated by a polymer scientist skilled in the art that the method of synthesis is immaterial, inasmuch as the produced polymer achieves at least one of the above defined parameters.

For example, U.S. Pat. No. '614 discloses different crosslinkers and crosslinker amounts that yield crosslinked PVP suitable for the present invention. The effect of crosslinker amount on swell volume and viscosity is graphically presented in Shih, J. S., et al. (1995). Thus, the crosslinked PVP may be produced by the precipitation polymerization method of the '614 patent, by the hydrogel method described in the '385 patent, or by the non-aqueous, heterogeneous polymerization method of the '068 patent. Certainly, other techniques are contemplated to synthesize this polymer, provided the product meets the aqueous swelling parameter and Brookfield viscosity requirements.

Final product viscosities may slightly vary for compositions containing crosslinked PVP made by these different methods. Nonetheless, these variations are within the scope of the invention, as the crosslinked PVPs thicken the disclosed oral care compositions.

Unless otherwise specified, “strongly swellable, lightly to moderately crosslinked PVP” does not refer to swellable but water-insoluble crosslinked PVP, such as the type sold into commercial trade under the trade name Polyclar® by International Specialty Products, which differs from the above described crosslinked PVP.

Commercially available examples of strongly swellable, lightly to moderately crosslinked PVP include, but are not limited to, FLEXITHIX™, ACP-1120, ACP-1179, and ACP-1180, available from International Specialty Products (Wayne, N.J.).

As recognized by one of ordinary skill in the art, the amount of thickening agent can vary depending upon the consistency and the desired thickness of the composition. In accordance with certain aspects of the present invention, the thickening agent may be used in amounts from about 0.5 to about 10%, more particularly from about 1% to about 7%, and in accordance with certain aspects of the invention from about 2% to about 5% by weight of the composition.

Compositions in accordance with certain aspects of the present invention may be formulated with various other excipients as known to those of ordinary skill in the art to improve performance.

The oral composition in the form of a dentifrice typically includes an orally acceptable vehicle including a water-phase with a humectant. Water is typically present in an amount of at least about 3% by weight, generally about 3-35% and a humectant, typically glycerine, xylitol and/or sorbitol, typically total about 6.5-75% or 80% by weight of the oral composition, more typically about 10-75%. Reference hereto to sorbitol refers to the material typically as available commercially in 70% aqueous solutions. The composition may include solubilizing agents, examples of which include humectant polyols such as propylene glycol, butylene glycol, polyethylene glycol, dipropylene glycol, and hexylene glycol, cellosolves such as methyl cellosolve and ethyl cellosolve, vegetable oils and waxes containing at least about 12 carbons in a straight chain such as olive oil, castor oil and petrolatum and esters such as amyl acetate, ethyl acetate and benzyl benzoate. As used herein “propylene glycol” includes 1,2-propylene glycol and 1,3-propylene glycol. Trimethyl glycine may also be used.

Suitable solvents for the present composition include water, edible polyhydric alcohols such as glycerin, diglycerin, triglycerin, sorbitol, xylitol, butylene glycol, erythritol, polyethylene glycol, propylene glycol, and combinations thereof.

The pH of the oral composition is generally in the range of about 4 to about 9 or 10. The pH can be controlled with acid (e.g. citric acid or benzoic acid) or base (e.g. sodium hydroxide) or buffered (as with sodium citrate, benzoate, carbonate, or bicarbonate, disodium hydrogen phosphate, sodium dihydrogen phosphate, etc.).

In one embodiment, the compositions disclosed herein are dentifrices in paste, gel or liquid forms. Components of such products generally include the present strongly swellable, lightly to moderately crosslinked polyvinyl pyrrolidone as thickening agent, one or more humectants (from 0% up to about 10%), one or more of a dental polishing agent (from about 6% to about 50%), a surfactant (from about 0.5% to about 10%), a flavoring agent (from about 0.04% to about 2%), a sweetening agent (from about 0.1% to about 3%), a coloring agent (from about 0.01% to about 0.5%) and water or other solvent (up to 90%). Dentifrices may also include one or more of an anticaries agent (such as from about 0.05% to about 0.3% as fluoride ion) and an anticalculus agent (from about 0.1% to about 13%).

Other embodiments of the subject invention are liquid products, including mouthwashes or rinses, mouth sprays, dental solutions and irrigation fluids wherein the thickening agent comprises a strongly swellable, lightly to moderately crosslinked polyvinyl pyrrolidone. Components of such mouthwashes and mouth sprays typically include one or more of water or other solvent (from about 45% to about 95%), ethanol (from about 0% to about 25%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion) and an anticalculus agent (from about 0.1% to about 3%). Components of dental solutions generally include one or more of water or other solvent (from about 90% to about 99%), preservative (from about 0.01% to about 0.5%), flavoring agent (from about 0.04% to about 2%), sweetening agent (from about 0.1% to about 3%), and surfactant (from 0% to about 5%).

In accordance with certain aspects of the present invention, the oral composition may contain a polishing material. Dental polishing materials useful in the compositions of the subject invention include many different materials. Suitable polishing materials include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of polishing materials for use in the present compositions is the particulate thermo-setting polymerized resins as described in U.S. Pat. No. 3,070,510 issued to Cooley et al. the contents of which are hereby incorporated by reference. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types are particularly useful because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The polishing material can be precipitated silica or silica gels such as the silica xerogels described in Pader et al., U.S. Pat. No. 3,538,230 and DiGiulio, U.S. Pat. No. 3,862,307, the contents of which are hereby incorporated by reference. Examples include the silica xerogels marketed under the trade name “Syloid” by the W.R. Grace & Company, Davison Chemical Division and precipitated silica materials such as those marketed by the J. M. Huber Corporation under the trade name, Zeodent®, particularly the silicas carrying the designation Zeodent®119, Zeodent® 118, Zeodent® 109 and Zeodent® 129. Particularly useful polishing materials include crystalline silica having particle sizes of up to about 5 microns, a mean particle size of up to about 1.1 microns, and a surface area of up to about 50,000 cm²/gm., silica gel or colloidal silica and complex amorphous alkali metal aluminosilicate.

When visually clear or opacified gels are employed, a polishing agent of colloidal silica, such as those sold under the trademark SYLOID as Syloid 72 and Syloid 74 or under the trademark SANTOCEL as Santocel 100 or alkali metal aluminosilicate complexes (that is, silica containing alumina combined in its matrix) are particularly useful, since they are consistent with gel-like texture and have refractive indices close to the refractive indices of gelling agent-liquid (including water and/or humectant) systems commonly used in dentifrices.

The polishing agent is generally present in the oral composition dentifrices such as toothpaste or gel compositions in weight concentrations of about 5% to about 70%, more particularly from about 10% to about 30%. Mixtures of polishing agents can be used.

In gel formulations, the liquid vehicle may typically comprise about 3-35% by weight of water, such as about 10-35%, and humectant in an amount ranging from about 6.5% to about 80%, such as about 10% to about 80% by weight of the preparation. In clear gels where the refractive index is an important consideration, about 3-30% of water, 0 to about 70% of glycerine and about 20-25% of sorbitol are preferably employed. In accordance with certain embodiments, water may comprise up to about 99% by weight of the aqueous compositions herein.

In addition to the polyvinyl pyrrolidone thickening agent, the oral composition may also contain other thickeners such as a natural or synthetic thickener or gelling agent typically in proportions of about 0.1 to about 10%, preferably about 0.5 to about 5%. Examples of these secondary thickeners include synthetic hectorite, a synthetic colloidal magnesium alkali metal silicate complex clay available for example as Laponite (e.g. CP, SP 2002,D) marketed by Laporte Industries Limited. Laponite D analysis shows, approximately by weight, 58.00% SiO₂, 25.40% MgO, 3.05% Na₂O, 0.98% Li₂O, and some water and trace metals. Its true specific gravity is 2.53 and it has an apparent bulk density (g./ml. at 8% moisture) of 1.0.

Other suitable gelling agents or thickeners include Irish moss, i-carrageenan, gum tragacanth, starch, polyvinylpyrrolidone, hydroxyethypropyl-cellulose, hydroxybutyl methyl cellulose, hydroxypropyl methyl cellulose, hydroxyethyl cellulose (e.g. available as Natrosol), sodium carboxymethyl cellulose, and colloidal silica such those available as finely ground Syloid 244 and Sylodent 15.

The oral composition may also contain a source of fluoride ions, or fluorine-providing component, as anti-caries agent, in an amount sufficient to supply about 25 ppm to 5,000 ppm of fluoride ions. These compounds may be slightly soluble in water or may be fully water-soluble. They are characterized by their ability to release fluoride ions in water and by substantial freedom from undesired reaction with other compounds of the oral preparation. Among these materials are inorganic fluoride salts, such as soluble alkali metal, alkaline earth metal salts, for example, sodium fluoride, potassium fluoride, ammonium fluoride, calcium fluoride, a copper fluoride such as cuprous fluoride, zinc fluoride, barium fluoride, sodium fluorosilicate, ammonium fluorosilicate, sodium fluorozirconate, ammonium fluorozirconate, sodium monofluorophosphate, aluminum mono-and di-fluorophosphate, and fluorinated sodium calcium pyrophosphate. Alkali metal and tin fluorides, such as sodium and stannous fluorides, sodium monofluorophosphate (MFP) and mixtures thereof, are preferred.

The amount of fluorine-providing compound is dependent to some extent upon the type of compound, its solubility, and the type of oral preparation, but it must be a non-toxic amount, generally about 0.0005 to about 3.0% in the preparation. hi a dentifrice preparation, e.g. dental gel and an amount of such compound which releases up to about 5,000 ppm of fluoride ion by weight of the preparation is considered satisfactory. Any suitable minimum amount of such compound may be used, but it is preferable to employ sufficient compound to release about 300 to 2,000 ppm, more preferably about 800 to about 1,500 ppm of fluoride ion.

Typically, in the cases of alkali metal fluorides, this component is present in an amount up to about 5% by weight, based on the weight of the preparation, and preferably in the range of about 0.05% to 1%. In the case of sodium monofluorophosphate, the compound may be present in an amount of about 0.1-3%, more typically about 0.3-0.6%.

It will be understood that, as is conventional, the oral preparations may be sold or otherwise distributed in suitable labelled packages. Thus a dentifrice gel will usually be in a collapsible tube, typically aluminum, lined lead or plastic, or other squeeze, pump or pressurized dispenser for metering out the contents, having a label describing it, in substance, as a dentifrice gel or the like.

Organic surface-active agents may be used in the compositions of the present invention to achieve increased prophylactic action. Moreover, they assist in achieving thorough and complete dispersion of the composition throughout the oral cavity, and render the instant compositions more cosmetically acceptable. The organic surface-active material is preferably anionic, nonionic or ampholytic in nature, and it is preferred to employ as the surface-active agent a detersive material which imparts to the composition detersive and foaming properties. Suitable examples of anionic surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine which should be substantially free from soap or similar higher fatty acid material. The use of these sarcosinate compounds in the oral compositions of the present invention is particularly advantageous since these materials exhibit a prolonged and marked effect in the inhibition of acid formation in the oral cavity due to carbohydrate breakdown in addition to exerting some reduction in the solubility of tooth enamel in acid solutions. Examples of water-soluble nonionic surfactants are condensation products of ethylene oxide with various reactive hydrogen-containing compounds reactive therewith having long hydrophobic chains (e.g. aliphatic chains of about 12 to 20 carbon atoms), which condensation products (“ethoxamers”) contain hydrophilic polyoxyethylene moieties, such as condensation products of poly(ethylene oxide) with fatty acids, fatty alcohols, fatty amides, polyhydric alcohols (e.g. sorbitan monostearate) and polypropyleneoxide (e.g. Pluronic materials).

Surface active agent is typically present in amounts of about 0.5-5% by weight, more particularly about 1-2.5%.

Various other materials may be incorporated in the oral preparations of this invention such as whitening agents, preservatives, silicones, chlorophyll compounds and/or ammoniated material such as urea, diammonium phosphate, and mixtures thereof. These adjuvants, where present, are incorporated in the preparations in amounts which do not substantially adversely affect the properties and characteristics desired.

Any suitable flavoring or sweetening material may also be employed. Examples of suitable flavoring constituents are flavoring oils, e.g. oil of spearmint, peppermint, wintergreen, sassafras, clove, sage, eucalyptus, marjoram, cinnamon, lemon, and orange, and methyl salicylate. Suitable sweetening agents include sucrose, lactose, maltose, xylitol, sodium cyclamate, perillartine, AMP (aspartyl phenyl alanine, methyl ester), saccharine and the like. Suitably, flavor and sweetening agents may each or together comprise from about 0.1% to 5% or more of the preparation.

The present compositions may optionally include active agents, such as antimicrobial/anti-plaque agents, tooth bleaching actives, dentinal desensitizing agents, etc. An anticalculus agent such as sodium tripolyphosphate, tetrapotassium or tetrasodium pyrophosphates, or mixtures thereof, can also be present in the composition, typically in concentrations from about 0.5 to about 8% by weight.

Examples of antimicrobial agents include, but are not limited to, zinc salts, stannous salts, cetyl pyridinium chloride, chlorhexidine, triclosan, triclosan monophosphate, and flavor oils. Examples of dentinal desensitizing agents to control hypersensitivity include, but are not limited to, salts of potassium, calcium, strontium and tin including nitrate, chloride, fluoride, phosphates, pyrophosphate, polyphosphate, citrate, oxalate and sulfate. Another method for controlling hypersensitivity involves the use of a mechanical shield around the nerve by blocking of the dentinal tubules wholly or partially with tubule blocking agents. Examples of tubule blocking agents include, but are not limited to, cationic alumina, clays, water-soluble or water-swellable polyelectrolytes, maleic acid copolymers and polyethylene particles.

Examples of tooth bleaching actives include, but are not limited to, PVP, peroxides, perborates, percarbonates, peroxyacids, persulfates, and combinations thereof. Suitable peroxide compounds include hydrogen peroxide, urea peroxide, calcium peroxide, carbamide peroxide, sodium peroxide, zinc peroxide and mixtures thereof. A preferred percarbonate is sodium percarbonate.

The compositions may also include an antibacterial enhancing agent (AEA) which enhances the delivery and retention of the antibacterial agent to, and retention thereof on oral surfaces. The AEA, when used, is typically present in an amount of about 0.05% to about 5%, more particularly from about 0.1% to about 3%. Examples of AEA's useful in the present invention are disclosed in U.S. Pat. Nos. 5,188,821 and 5,192,531; the contents of which are hereby incorporated by reference. Specific examples include synthetic anionic polymeric polycarboxylates, in the form 1:4 to 4:1 copolymers of maleic anhydride or acid with another polymerizable ethylenically unsaturated monomer, preferably methyl vinyl ether/maleic anhydride having a molecular weight (M.W.) of about 30,000 to about 1,000,000, most preferably about 30,000 to about 800,000. These copolymers are available for example as Gantrez®, e.g. AN 139 (M.W. 500,000), AN 119 (M.W. 250,000) and preferably S-97 Pharmaceutical Grade (M.W. 700,000) available from ISP. Other examples include mucoadhesive polymers and carboxylic acid containing copolymers.

Titanium dioxide may also be added to the present compositions as coloring or opacifying agent typically at a level of from about 0.25% to about 5% by weight.

The present invention also relates to methods for cleaning, refreshing and controlling bacterial activity in the oral cavity which cause undesirable conditions including plaque, caries, calculus, gingivitis, periodontal disease and malodor. The benefits of these compositions may increase over time when the composition is used repeatedly.

The method of use or treatment herein may comprise contacting a subject's dental enamel surfaces and mucosa in the mouth with the oral compositions disclosed herein. The method may comprise brushing with a dentifrice or rinsing with a dentifrice slurry or mouthrinse. Other methods include contacting the topical oral gel, denture product, mouthspray, or other form with the subject's teeth and oral mucosa. The subject may be any person or animal whose tooth surface and oral cavity are contacted with the oral composition. By animal is meant to include household pets or other domestic animals, or animals kept in captivity.

The compositions of this invention can be incorporated in lozenges, or in chewing gum or other products, e.g. by stirring into a warm gum base or coating the outer surface of a gum base, illustrative of which may be mentioned jelutong, rubber latex, vinylite resins, etc., desirably with conventional plasticizers or softeners, sugar or other sweeteners or carbohydrates such as glucose, sorbitol and the like.

The following examples are further illustrative of the nature of the present invention, but it is understood that the invention is not limited thereto. All amounts and proportions referred to herein and in the appended claims are by weight, unless otherwise indicated.

A variety of alternative formulations containing the viscosity builder of the present invention are easily extrudable, have acceptable organoleptic properties and remained stable over the span of this study. Two examples of specific compositions, each utilizing one of two common abrasives—silica and chalk (Calcium Carbonate)—are shown below:

### EXAMPLE 1

| Component | % |
|---|---|
| Water | q.s. to 100 |
| Viscosity Builder (ACP-1120) | 3.00 |
| Sodium Fluoride | 0.24 |
| Sodium Saccharin | 0.30 |
| Titanium Dioxide | 0.50 |
| 70% Sorbitol Solution | 27.00 |
| Glycerin | 10.00 |
| Hydrophilic Fumed Silica | 2.00 |
| Precipitated Synthetic Amorphous Silica | 20.00 |
| Flavor Mix | 2.00 |
| Sodium Lauryl Sulfate | 1.00 |

The composition in Example 1 can be prepared on a laboratory scale by adding water to an appropriately sized mixing vessel equipped with an overhead mixer and a propeller blade stirring shaft. Sodium fluoride and sodium saccharin are added to the mixing vessel and mixed until dissolved. Mixing speed is increased and the viscosity builder is added slowly to the mixing vessel to avoid lump formation. Mixing is continued for about 20 minutes or until the viscosity builder is fully dispersed and hydrated. Titanium is added and mixed until dispersed. The sorbitol solution and glycerin are added with mixing followed by the fumed silica and amorphous silica in that order. When the silicas are fully dispersed the flavor mix is added and mixed until incorporated. The composition is then transferred to a Whip Mix® Vacuum Power Mixer and mixed for 15 minutes under a vacuum of not less than 28 inches Hg. Sodium Lauryl Sulfate is then added to the mixing vessel and mixed under vacuum as described above for 15 to 20 minutes.

One skilled in the art can easily scale up this composition to commercial scale using no more than routine experimentation.

### EXAMPLE 2

| Component | % |
|---|---|
| Water | q.s. to 100 |
| Viscosity Builder (ACP-1120) | 3.00 |
| Sodium Fluoride | 0.24 |
| Sodium Saccharin | 0.30 |
| Titanium Dioxide | 0.50 |
| 70% Sorbitol Solution | 24.00 |
| Glycerin | 10.00 |
| Hydrophilic Fumed Silica | 2.00 |
| Chalk | 35.00 |
| Flavor Mix | 1.00 |
| Sodium Lauryl Sulfate | 1.00 |

The composition in Example 2 is prepared on a laboratory scale by adding water to an appropriately sized mixing vessel equipped with an overhead mixer and a propeller blade stirring shaft. Sodium fluoride and sodium saccharin are added to the mixing vessel and mixed until dissolved. Titanium dioxide is then added and mixed until well dispersed. Because of the low volume of water, sorbitol solution and glycerin are added to the mixing vessel at this point followed by the fumed silica. The chalk is then weighed out in a beaker. The viscosity builder is weighed out in a separate beaker of appropriate size. Chalk is then added to the beaker containing the viscosity builder such that the ratio of chalk to viscosity builder is approximately 5 to 1. The chalk /viscosity builder mixture is then dry blended by mixing with a spatula until the two components are uniformly dispersed. The mixing speed is increased and the dry blend is added to the mixing vessel slowly. Mixing is continued until the viscosity builder is fully hydrated and the chalk completely dispersed, The remaining chalk and the flavor mix are added at this point and mixed until well incorporated. The composition is then transferred to a Whip Mix Vacuum Power Mixer and mixed for 15 minutes under a vacuum of not less than 28 inches Hg. Sodium Lauryl Sulfate is then added to the mixing vessel and mixed under vacuum as described above for 15 to 20 minutes. One skilled in the art can easily scale up this composition to commercial scale using no more than routine experimentation.

After completion, aliquots of finished product can be taken for analyses which may include, but are not limited to, Brookfield viscosity, specific gravity, color, odor and extrudability. In these experiments, the compositions described above were compared to current commercially available toothpaste. To assess the ease of dispensing the compositions were placed in unused blank laminate toothpaste tubes. The tubes were then squeezed to extrude a ribbon of toothpaste. The ease of doing this was compared to that of the commercial products and was found to be comparable for both Example 1 and Example 2, The appearance of the extruded ribbons were visually assessed and were found to compare favorably to commercial toothpaste, Lastly, a ribbon of each product was applied to toothbrushes and used by two volunteers. After brushing it was agreed that both experimental compositions had acceptable mouth feel, foaming and abrasive characteristics.

What is claimed is:

1. An oral care composition comprising:
at least one oral care ingredient; and
a thickening agent, wherein the thickening agent comprises a strongly swellable, lightly to moderately crosslinked polyvinyl pyrrolidone.

2. An oral care composition according to claim 1 in the form of a dentifrice.

3. An oral care composition according to claim 1 wherein the thickening agent is characterized by an aqueous gel volume of about 15 to 150 ml/g of polymer and a Brookfield viscosity of at least 10,000 cps for a 5% aqueous solution at 25° C.

4. An oral care composition according to claim 1 wherein the oral care ingredient is selected from the group consisting of a polishing agent, fluoride ion source, humectant, surface active agent and combinations thereof.

5. An oral care composition according to claim 4 wherein said oral care composition comprises a polishing agent.

6. An oral care composition according to claim 5 wherein said polishing agent is selected from the group consisting of silicas, insoluble sodium polymetaphosphate, hydrated alumina, calcium carbonate, dicalcium orthophosphate dihydrate, calcium pyrophosphate, tricalcium phosphate, calcium polymetaphosphate, resinous abrasive materials, and mixtures thereof.

7. An oral care composition according to claim 1 wherein the composition is in the form of a gel.

8. An oral care composition according to claim 7 wherein the composition comprises a source of fluoride ions selected from the group consisting of sodium fluoride, potassium fluoride, ammonium fluoride, calcium fluoride, copper fluoride, zinc fluoride, barium fluoride, sodium fluorosilicate, ammonium fluorosilicate, sodium fluorozirconate, ammonium fluorozirconate, sodium monofluorophosphate, aluminum mono-and di-fluorophosphate, fluorinated sodium calcium pyrophosphate and combinations thereof.

9. An oral care composition comprising a dentifrice wherein said dentifrice comprises a source of fluoride ions and a thickening agent, wherein the thickening agent comprises a strongly swellable, lightly to moderately crosslinked polyvinyl pyrrolidone.

10. The oral care composition of claim 9 wherein the concentration of the thickening agent is present in an amount from about 0.5 to about 10% by weight.

11. The oral care composition of claim 9 wherein the thickening agent is characterized by an aqueous gel volume of about 15 to 150 ml/g of polymer and a Brookfield viscosity of at least 10,000 cps for a 5% aqueous solution at 25° C.

12. The oral care composition of claim 11 wherein the composition further comprises at least one of a polishing agent, an oral care active agent, an anticaries agent, an anticalculus agent and a humectant.

13. The oral care composition of claim 12 wherein the oral care composition comprises:
from 0% up to about 10% of a humectant;
from about 6% to about 50% of a polishing agent;
from about 0.5% to about 10% of a surfactant;
from about 0.04% to about 2% of a flavoring agent or sweetening agent;
and up to about 90% water.
